(19) United States
(12) Patent Application Publication
(10) Pub. No.: US 2026/0007644 A1
(43) Pub. Date: Jan. 8, 2026

(54) NIMODIPINE COMPOSITION FREE OF ETHANOL AND PHOSPHOLIPID FOR MOIST HEAT STERILIZATION AND METHOD FOR PREPARING SAME

(71) Applicant: BEIJING DELIVERY PHARMACEUTICAL TECHNOLOGY CO., LTD, Beijing (CN)

(72) Inventors: Cuishuan WU, Beijing (CN); Dan ZHANG, Beijing (CN); Yamei SUN, Beijing (CN); Shuangyue LOU, Beijing (CN)

(21) Appl. No.: 18/878,342

(22) PCT Filed: Jun. 25, 2023
(86) PCT No.: PCT/CN2023/102147

§ 371 Date: May 1, 2025

(30) Foreign Application Priority Data
Jun. 27, 2022 (CN) 202210734212.7

(51) Int. Cl. A61K 31/4422; A61K 9/08; A61K 47/10; A61K 47/26; A61K 47/34; A61K 47/44; A61L 2/00

(52) U.S. Cl. CPC A61K 31/4422; A61K 9/08; A61K 47/10; A61K 47/26; A61K 47/34; A61K 47/44; A61L 2/0023; A61L 2202/21

(57) ABSTRACT

Provided are a moist-heat sterilized nimodipine composition free of ethanol and phospholipid and a method for preparing the same. The composition comprises (1) nimodipine; (2) a non-phospholipid surfactant selected from polyoxyethylene castor oil, polyoxyethylene hydrogenated castor oil, polyoxyl 15 hydroxystearate, D-alpha-tocopherol polyethylene glycol 1000 succinate (TPGS), polysorbates, glyceryl monocaprylocaprate, or any mixture of two or more thereof; and (3) a co-solvent, which is propylene glycol.

The present application claims the priority of the Chinese patent application CN202210734212.7 filed on Jun. 27, 2022, the entire contents of which is hereby incorporated by reference.

### TECHNICAL FIELD

The present invention pertains to the field of pharmaceutical formulations. More particularly, the present application relates to a moist-heat sterilized nimodipine composition free of ethanol and phospholipid and a method for preparing the same.

### BACKGROUND

Nimodipine with the chemical name 1-methylethyl 2-methoxyethyl (±)-2,6-dimethyl-4-(3-nitrophenyl)-1,4-dihydropyridine-3,5-dicarboxylate is light yellow crystalline powder insoluble in water, and has a molecular formula of C₂₁H₂₆N₂O₇ and a molecular weight of 418.44. The structural formula is:

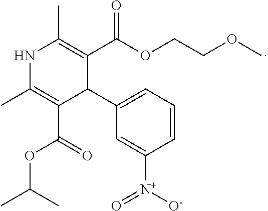

Nimodipine is a 1,4-dihydropyridine calcium ion antagonist, exhibits high selectivity on the receptors in brain tissue, and can easily penetrate through the blood-brain barrier. By effectively preventing calcium ions from entering the cells and inhibiting smooth muscle contraction, nimodipine can achieve the purpose of relieving vasospasm, thereby protecting brain neurons and stabilizing their functions, enhancing cerebral blood perfusion, improving cerebral blood supply, and improving tolerance to hypoxia. Several studies have shown that nimodipine can effectively prevent and treat cerebral ischemic injury caused by cerebral vasospasm due to subarachnoid hemorrhage, reduce erythrocyte fragility and blood viscosity, inhibit platelet aggregation, and resist thrombosis. Nimodipine at the appropriate dose selectively dilates the cerebral vessels and almost does not affect the peripheral blood vessels, and can be used to improve the blood circulation in the recovery period of acute cerebrovascular diseases. Nimodipine can also improve memory disorders in patients with senile brain injury, so it can be used to prevent senile dementia and effectively improve cognitive function after stroke. Nimodipine also has a certain therapeutic effect on sudden deafness. Currently, nimodipine has been approved against a variety of indications, for example, for preventing and treating cerebral vasospasm after subarachnoid hemorrhage due to various causes, for improving the blood circulation in the recovery period of acute cerebrovascular diseases, for treating senile brain dysfunction, such as memory loss, orientation and attention disorders, and emotional fluctuation, for treating sudden deafness, and for treating mild and severe hypertension.

There are many dosage forms of nimodipine available on the market in China and other countries. The oral dosage forms include ordinary tablets, sustained release tablets, hard capsules, and soft capsules. The injections include ampoules and infusions. For their low solubility in the gastrointestinal fluids and the significant first pass effect in liver, the solid formulation of nimodipine has the disadvantage of poor bioavailability. The injection dosage forms of nimodipine have a faster onset and better therapeutic effect than the oral formulations thereof, so they are more widely used in clinical practice. However, there are significant defects in currently clinically applied nimodipine injections:
(1) Nimodipine injection contains a large amount of an organic solvent. Since nimodipine is poorly soluble in water, the commercially available infusion (50 mL: 10 mg, Nimotop®) contains 23.7% (v/v) ethanol (1 mL of the drug solution contains 200 mg of ethanol) and 17% (v/v) polyethylene glycol 400 so that the organic solvents account for 40% (20 mL). The ampoules contain a higher proportion of ethanol. The presence of the large amount of ethanol causes significant adverse effects of intravenous injection: the injections exhibit strong vascular irritation, tend to cause pain at the injection site when injected into the blood vessel, and tend to cause phlebitis of the injected blood vessel after repeated injections. In addition, ethanol has influence on nervous system, causing muscle incoordination, slow response, inattention, decreased self-control, memory loss, diminished intelligence and the like. Ethanol also causes damage to the liver and irritation to the stomach. The ethanol-containing formulations cannot be used in patients allergic to ethanol, and thus have a limited application in those patients. In addition, when the ethanol-containing formulation is administrated in combination with cephalosporins or nitroimidazoles, disulfiram-like adverse reactions will probably occur, and chest pain, myocardial infarction, acute heart failure, dyspnea, acute liver injury, convulsion and even death may be possibly induced.
(2) Nimodipine belongs to dihydropyridine derivatives. In order to control the quality of the drug products of nimodipine, the control of impurities, particularly Impurity I (2-methoxyethyl isopropyl 2,6-dimethyl-4-(3-nitrophenyl)-3,5-pyridinedicarboxylate) of nimodipine formulations is required in pharmacopoeias of many countries including Chinese Pharmacopoeia and European Pharmacopoeia. Impurity I as the main impurity for nimodipine degradation is a structural analogue of compounds with hepatorenal toxicity, poses a safety risk, and thus is the major impurity for quality control. The content of Impurity I in the injections is as lower as better. Impurity I is generated by dehydrogenation of the dihydropyridine ring of nimodipine. The formation of this impurity could be promoted by various conditions, including strong acid, strong base, elevated temperature, oxidation, light and the like. The conventional nimodipine injections contain citrate buffer as a pH regulator, which can prevent the pH value of the injections from fluctuating. However, the generation of impurities in the manufacture process could not be well controlled merely by the stabilization of the pH value.

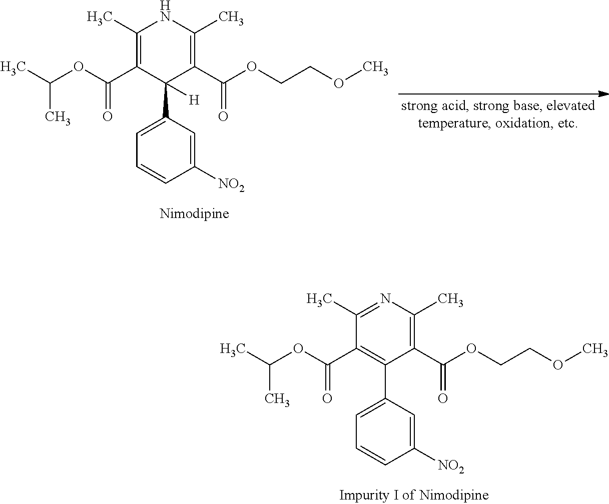

(3) Nimodipine must be infused slowly at a rate of 1-2 mg/h, and the time required for the infusion of 10 mg nimodipine is at least 5 hours, otherwise the side effects could not be tolerated by the patients. The originator's product Nimotop® uses a high concentration of ethanol for solubilization, and is diluted with dextrose injection or sodium chloride injection for use, resulting in a decrease in the concentration of ethanol and crystallization upon standing. In order to solve the problem of poor physical stability caused by the dilution, it is necessary to use a special three-way valve infusion set (see FIG. 1), which brings inconvenience in clinical use.

Accordingly, there is a need to develop a novel nimodipine composition free of ethanol, which can also ensure the good stability of nimodipine.

### SUMMARY OF THE INVENTION

After doing a lot of experiments, the inventors surprisingly found that the clinically applicable dosage of nimodipine can be prepared into a more stable composition free of ethanol by preparing a liquid concentrate using a non-phospholipid surfactant and a specific co-solvent and performing moist heat sterilization. The composition can achieve good dissolution of nimodipine, and thus is a homogenous transparent solution, and the preparation method thereof is extremely simple. The composition may be diluted with an aqueous vehicle to form a solution prior to use, and the formed solution can be directly used for administration conveniently, has excellent stability without the problem of crystallization. The aqueous vehicles may be aqueous vehicles for injection (e.g., water for injection, 5% dextrose injection, 0.9% sodium chloride injection, lactated Ringer's solution, dextran 40 solution, hydroxyethyl starch 130/0.4 in sodium chloride injection, hydroxyethyl starch 200/0.5 in sodium chloride injection, etc.) or vehicles suitable for oral administration (e.g., purified water, etc.). Moreover, the composition in accordance with the invention fully meets the requirements for intravenous injection and can be administered by infusion through an ordinary infusion set in light of its excellent stability after dilution. Therefore, the composition in accordance with the invention not only has excellent stability, but also can be easily administered, particularly by injection, and satisfies the unmet clinical needs.

The purpose of the invention is to provide a moist-heat sterilized nimodipine composition free of ethanol and phospholipid, and a method for preparing the same, which is simple, environment-friendly, and applicable in industry.

In the first aspect, the invention provides a moist-heat sterilized nimodipine composition free of ethanol and phospholipid, characterized in that the composition comprises:
(1) nimodipine;
(2) a non-phospholipid surfactant selected from polyoxyethylene castor oil, polyoxyethylene hydrogenated castor oil, polyoxyl 15 hydroxystearate, D-alpha-tocopherol polyethylene glycol 1000 succinate (TPGS), polysorbates, glyceryl monocaprylocaprate, or any mixture of two or more thereof;
(3) a co-solvent, which is propylene glycol,
wherein, based on the total weight of nimodipine, the non-phospholipid surfactant and the co-solvent, the amount of nimodipine is ≤1.1% w/w, preferably 0.5%-1.1% w/w, more preferably 0.8-1.1% w/w; the amount of the non-phospholipid surfactant is 42-50% w/w, preferably 44-46% w/w; and the remainder is the co-solvent, and
the composition is terminally sterilized by moist heat sterilization.

The composition in accordance with the invention may further comprise a pH regulator and an antioxidant. The pH regulator may be selected from one or more of citric acid, citrate (e.g., sodium citrate), maleic acid, tartaric acid, hydrochloric acid, sodium hydroxide, acetic acid, acetate (e.g., sodium acetate), phosphoric acid, phosphate (e.g., sodium hydrogen phosphate, sodium dihydrogen phosphate, or sodium phosphate). The antioxidant may be selected from one or more of α-tocopherol succinate, ascorbyl palmitate, butylated hydroxyanisole (BHA), butylated hydroxytoluene (BHT).

In a preferred embodiment of this aspect, the composition in accordance with the invention consists of nimodipine, the non-phospholipid emulsifier and the co-solvent.

In the second aspect, the present invention provides a method for preparing the composition in accordance with the invention, characterized in that the method comprises the steps of: (1) mixing nimodipine, the non-phospholipid surfactant and the co-solvent to form a solution, if necessary, with heating to dissolve, (2) filling the resultant solution into a container, and (3) subjecting the filled solution to moist heat sterilization,
optionally, the method is performed with protection from light,
optionally, purging the solution obtained in step (1) with nitrogen gas prior to step (2),
preferably, the moist heat sterilization is performed at 115° C. for 30 min or at 121° C. for 15 min.

The composition in accordance with the invention may be diluted with an aqueous vehicle to form a solution. The formed solution is a homogenous transparent solution, which has good stability, and can be administered by intravenous infusion through an ordinary infusion set.

Briefly, the composition free of ethanol and phospholipid of the present invention has the following advantages: (1) the composition can dissolve nimodipine well, avoid the irritation caused by ethanol, satisfy the need of patients allergic to ethanol, and avoid the disulfiram-like adverse reaction, which will probably occur in the combination therapy; (2) the composition can be subjected to the moist heat sterilization, and ensure both the good sterile status and the low impurity content, particularly the significantly lower content of Impurity I with hepatorenal toxicity; (3) the solution obtained by diluting the composition with an aqueous vehicle has good stability and could be administered by infusion through an ordinary infusion set. Therefore, the composition in accordance with the invention has good safety and stability, is convenient for clinical use, and provides a better choice for the clinical application of nimodipine.

### DETAILED DESCRIPTION

### Specific Embodiments

Embodiment 1. A nimodipine composition free of phospholipid and ethanol, characterized in that the composition comprises:
(1) nimodipine;
(2) a non-phospholipid surfactant selected from polyoxyethylene castor oil, polyoxyethylene hydrogenated castor oil, polyoxyl 15 hydroxystearate, D-alpha-tocopherol polyethylene glycol 1000 succinate (TPGS), polysorbates, glyceryl monocaprylocaprate, or any mixture of two or more thereof;
(3) a co-solvent, which is propylene glycol,
wherein, based on the total weight of nimodipine, the non-phospholipid surfactant and the co-solvent, the amount of nimodipine is ≤1.1% w/w, preferably 0.5%-1.1% w/w, more preferably 0.8-1.1% w/w; the amount of the non-phospholipid surfactant is 42-50% w/w, preferably 44-46% w/w; and the remainder is the co-solvent, and
the composition is terminally sterilized by moist heat sterilization.

Embodiment 2. The composition according to Embodiment 1, wherein the non-phospholipid surfactant is selected from polyoxyl 35 castor oil, pure polyoxyl 35 castor oil, polyoxyl 40 hydrogenated castor oil, polyoxyl 60 hydrogenated castor oil, polyoxyl 15 hydroxystearate, D-alpha-tocopherol polyethylene glycol 1000 succinate (TPGS), polysorbate 20, polysorbate 21, polysorbate 40, polysorbate 60, polysorbate 61, polysorbate 65, polysorbate 80, polysorbate 81, polysorbate 85, polysorbate 120, glyceryl monocaprylocaprate, or any mixture of two or more thereof.

Embodiment 3. The composition according to Embodiment 2, wherein the non-phospholipid surfactant is polyoxyl 15 hydroxystearate.

Embodiment 4. The composition according to any one of Embodiments 1 to 3, wherein the composition further comprises a pH regulator and an antioxidant, and wherein the pH regulator is preferably selected from one or more of citric acid, citrate (e.g., sodium citrate), maleic acid, tartaric acid, hydrochloric acid, sodium hydroxide, acetic acid, acetate (e.g., sodium acetate), phosphoric acid, and phosphate (e.g., sodium hydrogen phosphate, sodium dihydrogen phosphate, or sodium phosphate), the antioxidant is preferably selected from one or more of α-tocopherol succinate, ascorbyl palmitate, butylated hydroxyanisole, and butylated hydroxytoluene.

Embodiment 5. The composition according to any one of Embodiments 1 to 3, wherein the composition consists of nimodipine, the non-phospholipid surfactant and the co-solvent.

Embodiment 6. The composition according to Embodiment 5, wherein the composition consists of nimodipine, polyoxyl 15 hydroxystearate and propylene glycol, and wherein based on the total weight of the composition, the amount of nimodipine is ≤1.1% w/w, preferably 0.5%-1.1% w/w, more preferably 0.8-1.1% w/w; the amount of polyoxyl 15 hydroxystearate is 42-50% w/w, preferably 44-46% w/w; and the remainder is propylene glycol, and the composition is terminally sterilized by moist heat sterilization.

Embodiment 7. The composition according to any one of Embodiments 1 to 6, wherein the composition is prepared with protection from light.

Embodiment 8. The composition according to any one of Embodiments 1 to 7, wherein the composition is prepared by a method comprising a step of purging with nitrogen gas.

Embodiment 9. A method for preparing the composition according to any one of Embodiments 1 to 5, comprising the steps of: (1) mixing nimodipine, the non-phospholipid surfactant and the co-solvent to form a solution, if necessary, with heating to dissolve, (2) filling the resultant solution into a container, and (3) subjecting the filled solution to moist heat sterilization,
optionally, the method is performed with protection from light,
optionally, purging the solution obtained in step (1) with nitrogen gas prior to step (2),
preferably, the moist heat sterilization is performed at 115° C. for 30 min or at 121° C. for 15 min.

Embodiment 10. Use of the composition according to any one of Embodiments 1 to 8 in the manufacture of a solution for intravenous injection, in particular for intravenous infusion.

Embodiment 11. A solution obtained by diluting the composition according to any one of Embodiments 1 to 8 with an aqueous vehicle, wherein the aqueous vehicle is preferably a vehicle suitable for injection, for example, water for injection, 5% dextrose injection, 0.9% sodium chloride injection, lactated Ringer's solution, dextran 40 solution, hydroxyethyl starch 130/0.4 in sodium chloride injection, or hydroxyethyl starch 200/0.5 in sodium chloride injection.

Embodiment 12. The solution according to Embodiment 11, which is used for intravenous injection, in particular for intravenous infusion.

The Composition in Accordance with the Invention and the Method for Preparing the Same

Through further study on the basic physical and chemical properties of nimodipine, the inventors found that if phospholipid is present in the prepared liquid concentrate containing nimodipine as the active ingredient, the liquid concentrate can only be sterilized by filtration, but not by moist heat sterilization, since the moist heat sterilization will induce the discoloration of the liquid concentrate, which makes the liquid concentrate fail to pass the stability test. To solve this problem, the inventors further studied and optimized the components of the composition, and obtained the composition in accordance with the invention.

The nimodipine composition in accordance with the invention is a liquid concentrate free of ethanol and phospholipid, and is terminally sterilized by moist heat sterilization. Prior to use, the liquid concentrate is diluted by an aqueous vehicle (for example, water for injection, 5% dextrose injection, 0.9% sodium chloride injection, lactated Ringer's solution, dextran 40 solution, hydroxyethyl starch 130/0.4 in sodium chloride injection, or hydroxyethyl starch 200/0.5 in sodium chloride injection) to form a solution, which has good physical and chemical stability and can be administered directly to the patient, for example, orally or by injection such as intravenous injection. Moreover, thanks to the good stability of the formed diluted solution, there is no need to use a three-way valve infusion set during intravenous infusion.

Therefore, in the first aspect, the present invention provides a nimodipine composition free of ethanol and phospholipid, characterized in that the composition comprises:
(1) nimodipine;
(2) a non-phospholipid surfactant;
(3) a co-solvent, which is propylene glycol,
wherein, based on the total weight of nimodipine, the non-phospholipid surfactant and the co-solvent, the amount of nimodipine is ≤1.1% w/w, preferably 0.5%-1.1% w/w, more preferably 0.8-1.1% w/w; the amount of the non-phospholipid surfactant is 42-50% w/w, preferably 44-46% w/w; and the remainder is the co-solvent, and
the composition is terminally sterilized by moist heat sterilization.

The non-phospholipid surfactant may be selected from polyoxyethylene castor oil (for example, polyoxyl 35 castor oil, especially pure polyoxyl 35 castor oil), polyoxyethylene hydrogenated castor oil (for example, polyoxyl 40 hydrogenated castor oil, polyoxyl 60 hydrogenated castor oil), polyoxyl 15 hydroxystearate, D-alpha-tocopherol polyethylene glycol 1000 succinate (TPGS), polysorbate (for example, polysorbate 20, 21, 40, 60, 61, 65, 80, 81, 85, 120, especially polysorbate 80), glyceryl monocaprylocaprate, or any mixture of two or more thereof. More preferably, the non-phospholipid surfactant is selected from polyoxyl 40 hydrogenated castor oil, polyoxyl 35 castor oil, pure polyoxyl 35 castor oil, polyoxyl 15 hydroxystearate, polysorbate 80, or any mixture of two or more thereof. More preferably, the non-phospholipid surfactant is polyoxyl 15 hydroxystearate.

The composition in accordance with the invention is a liquid concentrate, which may be diluted by an aqueous vehicle to form a solution. The aqueous vehicle may be an injectable vehicle (for example, water for injection, 5% dextrose injection, 0.9% sodium chloride injection, lactated Ringer's solution, dextran 40 solution, hydroxyethyl starch 130/0.4 in sodium chloride injection, or hydroxyethyl starch 200/0.5 in sodium chloride injection, etc.) or a vehicle for oral administration (for example, purified water, etc.). After dilution with a vehicle for injection, for example, 5% dextrose injection, the composition in accordance with the invention forms a solution that meets the requirements for intravenous injections, even for intravenous infusions. Thus, the diluted solution can be directly used for injection, such as subcutaneous injection, intradermal injection, intraperitoneal injection, and intravenous injection, including intravenous bolus injection and intravenous infusion.

The composition in accordance with the invention is a homogenous transparent solution, and has good physical and chemical stability. When it is filled in a colored bottle and tested at the temperature of 60° C. or illumination of 5000 Lux, the composition in accordance with the invention met the requirements on stability. Furthermore, a solution obtained by diluting the composition in accordance with the invention also has good physical and chemical stability. For example, after a solution obtained by diluting the composition in accordance with the invention with an aqueous vehicle such as 5% dextrose injection or 0.9% sodium chloride injection was left to stand at room temperature for 48 hours, no drug precipitation was observed.

The inventors screened the non-phospholipid surfactant used in the composition in accordance with the invention, and tested polyoxyl 40 hydrogenated castor oil (for example, kolliphor RH40), polyoxyl 15 hydroxystearate (for example, kolliphor HS15), D-alpha-tocopherol polyethylene glycol succinate (TPGS), and polysorbate 80 (for example, Tween 80). Experimental results showed that all these surfactants can solubilize nimodipine.

The inventors screened the co-solvent, including propylene glycol and glycerol. Experimental results showed that the use of propylene glycol as the co-solvent resulted in a homogeneous transparent solution of nimodipine, but the use of glycerol as the co-solvent made the formulated liquid concentrate layer upon standing.

The composition in accordance with the invention described above may consist of nimodipine, the non-phospholipid surfactant, and the co-solvent which is propylene glycol.

Alternatively, the nimodipine composition in accordance with the invention described above may further comprise, for example, a pH regulator and/or an antioxidant. The pH regulator may be selected from one or more of citric acid, citrate (e.g., sodium citrate), maleic acid, tartaric acid, hydrochloric acid, sodium hydroxide, acetic acid, acetate (e.g., sodium acetate), phosphoric acid, and phosphate (e.g., sodium hydrogen phosphate, sodium dihydrogen phosphate, or sodium phosphate). The antioxidant may be selected from one or more of α-tocopherol succinate, ascorbyl palmitate, butylated hydroxyanisole (BHA), and butylated hydroxytoluene (BHT).

The liquid concentrate in accordance with the invention and the diluted solution prepared therefrom can be used for preventing and treating cerebral vasospasm after subarachnoid hemorrhage due to various causes, for improving the blood circulation in the recovery period of acute cerebrovascular diseases, for treating senile brain dysfunction, such as memory loss, orientation and attention disorders, and emotional fluctuation, for treating sudden deafness, and for treating mild and severe hypertension.

In the second aspect, the present invention provides a method for preparing the composition in accordance with the invention, characterized in that the method is performed by: (1) mixing nimodipine, the non-phospholipid surfactant and the co-solvent to form a solution, if necessary, with heating to dissolve, (2) filling the resultant solution into a container, and (3) subjecting the filled solution to moist heat sterilization.

Since light may induce degradation of nimodipine, the method is preferably performed with protection from light, which can further reduce the related substances.

Moreover, the protection with nitrogen gas is also helpful to reduce the related substances of the liquid concentrate in accordance with the invention. Thus, preferably, the solution obtained in step (1) is purged with nitrogen gas prior to step (2).

The moist heat sterilization described herein may be performed under known conditions, for example, at 115° C. for 30 min or at 121° C. for 15 min.

The preparation method in accordance with the invention is simple, can be easily performed, and is suitable for industrial manufacture.

### Definitions

In the context of the present application, the terms “moist-heat-sterilized nimodipine composition free of ethanol and phospholipid”, “the composition in accordance with the invention”, “liquid concentrate in accordance with the invention”, “nimodipine composition in accordance with the invention” or its variants may be used interchangeably, and all refer to a composition comprising nimodipine, a non-phospholipid surfactant and a co-solvent, unless the context clearly indicates otherwise.

The term “polyoxyethylene castor oil” used herein refers to the substances obtained by reacting varying amounts of ethylene oxide and castor oil. Examples of polyoxyethylene castor oil include, but are not limited to, polyoxyl 35 castor oil, pure polyoxyl 35 castor oil.

The term “polyoxyl 35 castor oil” used herein refers to the substance obtained by reacting 1 mol of glycerol ricinoleate with 35 mol of ethylene oxide. It contains a small amount of polyethylene glycol ricinoleate and free ethylene glycol in addition to polyoxyethylene glycerol triricinoleate. Polyoxyl 35 castor oil is commercially available, for example from BASF or the like under the tradenames of kolliphor EL and kolliphor ELP.

The term “pure polyoxyl 35 castor oil” used herein refers to purified polyoxyethylene glycerol triricinoleate.

The term “polyoxyethylene hydrogenated castor oil” used herein refers to the substances obtained by reacting varying amounts of ethylene oxide and hydrogenated castor oil. Examples of polyoxyethylene hydrogenated castor oil include, but are not limited to, polyoxyl 40 hydrogenated castor oil, polyoxyl 60 hydrogenated castor oil.

The term “polyoxyl 40 hydrogenated castor oil” used herein refers to the substance obtained by reacting 1 mol of glycerol trihydroxystearic acid with 40-45 mol of ethylene oxide. It contains a small amount of polyethylene glycol trihydroxystearic acid and free ethylene glycol in addition to polyoxyethylene glycerol trihydroxystearate. Polyoxyl 40 hydrogenated castor oil is commercially available, for example from BASF or the like under the tradename of kolliphor RH40.

The term “polyoxyl 60 hydrogenated castor oil” used herein refers to the substance obtained by reacting 1 mol of glycerol trihydroxystearic acid with 60 mol of ethylene oxide. It contains a small amount of polyethylene glycol trihydroxystearic acid and free ethylene glycol in addition to polyoxyethylene glycerol trihydroxystearate.

The term “polyoxyl 15 hydroxystearate” used herein is a mixture formed by ethoxylation of 12-hydroxystearric acid, comprising mono-polyethylene glycol 12-hydroxystearate, di-polyethylene glycol 12-hydroxystearate and free polyethylene glycol as the main components, which has a condensation point of 22-30° C., is light yellow to white viscous semi-solid at room temperature, and turns to liquid at about 30° C. Polyoxyl 15 hydroxystearate is a novel non-ionic solubilizer and emulsifier, and is commercially available, for example, from BASF or Sigma-Aldrich under the tradename of kolliphor HS15 or Solutol HS-15. Polyoxyl 15 hydroxystearate has been recorded in German, British, American and European pharmacopoeias, and has the chemical name polyoxyl 15 hydroxystearate and macrogol 15 hydroxystearate respectively in the American pharmacopoeia and the European pharmacopoeia.

The term “D-alpha-tocopherol polyethylene glycol 1000 succinate (TPGS)” used herein is a water-soluble derivative of vitamin E, which is obtained by reaction between the carboxyl group of vitamin E succinate and the hydroxyl group of polyethylene glycol, and is commercially available, for example, under the tradename of Tocofersolan (TPGS) from BASF.

The term “polysorbates” used herein refers to a series of partial fatty acid esters of polyethoxylated sorbitan, which is formed by copolymerization of sorbitol and ethylene oxide in the ratio of 1 mol sorbitol to 20.5 mol or 4 mol ethylene oxide. Examples of polysorbates include, but are not limited to, for example, polysorbate 20, 21, 40, 60, 61, 65, 80, 81, 85, 120, particularly polysorbate 80. Polysorbates are commercially available, for example, under the tradenames of Tween 20, Tween 40, Tween 80, and the like from Nanjing Well Pharmaceutical Group Co., Ltd.

The term “glyceryl monocaprylocaprate” used herein refers to a mixed glycerol esters of medium chain fatty acids in vegetable oils, typically made from coconut oil and glycerol, and is commercially available, for example, under the tradename of IMWITOR742 from German IOI Oleo GmbH.

Unless otherwise specified, the percentages given herein are by weight (w/w).

### EXAMPLES

The following examples are intended to illustrate the invention, but not to limit in any way the scope thereof as defined by the appended claims.

The meanings of the abbreviations used in the examples are shown in Table 1.

TABLE 1
The meanings of abbreviations

| Abbreviations | Meaning | Sources |
|---|---|---|
| ELP | Polyoxyl 35 castor oil kolliphor EL | BASF |
| EPC | Egg Yolk lecithin EPC80 | Lipoid |
| RH40 | Polyoxyl 40 hydrogenated castor oil kolliphor RH40 | BASF |
| HS15 | Polyoxyl 15 hydroxystearate kolliphor HS15 | BASF |
| Tween 80 | Tween 80, polysorbate 80 | Nanjing Well |
| | | Pharmaceutical Group |
| TPGS | D-alpha-tocopherol polyethylene glycol 1000 | BASF |
| | succinate | |

### Example 1: Effects of Phospholipids on the Quality of Nimodipine Injection Before and After Sterilization

### Formulas 1-4: Effects of Phospholipids on the Quality of Nimodipine Injection Before and After Sterilization

TABLE 2
Effects of phospholipids on the quality of nimodipine
injection before and after sterilization

| Components (g) | Formula 1 | Formula 2 | Formula 3 |
|---|---|---|---|
| nimodipine | 1.00 | 1.00 | 1.00 |
| EPC | 0.00 | 2.00 | 10.00 |
| propylene glycol | 45.00 | 45.00 | 45.00 |
| HS15 | 54.00 | 54.00 | 54.00 |
| Total | 100.00 | 100.00 | 100.00 |

### Processes:

1) The prescribed amount of HS15 was added into a conical flask, heated at 60° C. until HS15 completely melted. The solution was shaken up and down to mix well for use;
2) The prescribed amount of EPC was weighed into another conical flask, and was added with the prescribed amounts of nimodipine as the active pharmaceutical ingredient (API), propylene glycol and the above heat-melted HS15 orderly. The flask was sealed followed by stirring in a water bath at 60° C. until all the components were dissolved to form a homogeneous oily solution. The resultant solution was filled into vials to give liquid concentrates of nimodipine containing different percentages of EPC;
3) The liquid concentrates of Formulas 1-3 were divided into two groups. One group was sterilized at 121° C. for 15 minutes, and the other group was not subjected to any sterilization treatment.

The liquid concentrates were examined for description, pH, peroxide value, free fatty acid, API content and the related substances before and after sterilization. The methods for detecting the description, pH, API content and the related substances were described in Example 11, and the methods for detecting the peroxide value and the free fatty acid were as follows.

Detection of free fatty acid: 5 g test solution was weighed accurately into a 25 ml volumetric flask, was diluted with isopropanol to the scale, and shaken evenly. 5 ml solution was accurately measured and added into a 20 ml tube with a stopper, was added with 5.0 ml the mixed solution of isopropanol:n-heptane:0.5 mol/L sulfuric acid (40:10:1), was shaken for 1 min and left to stand for 10 min. 3 ml n-heptane and 3 ml water were added accurately, sealed with a stopper, shaken up and down for 10 times, and left to stand for at least 15 minutes for layering. 3 ml upper layer solution was taken accurately into a 10 ml centrifuge tube, added with 1 ml Nile blue indicator solution (0.04 g Nile blue was dissolved in 200 ml water, and added with 100 ml n-heptane. The mixture was shaken, and n-heptane as the upper layer was discarded. These procedures were repeated four times. 20 ml aqueous solution from the lower layer was taken, added with 180 ml absolute ethanol, mixed well, and filled into a brown bottle. The prepared Nile blue indicator solution should be used within 1 month at room temperature), titrated with sodium hydroxide titration solution (0.01 mol/L) until the solution was light purple. The volumes of the titration solution in milliliters consumed by each batch of the test solution were compared.

Detection of peroxide value: 5 g sample was weighed accurately and added with 30 ml glacial acetic acid and chloroform (3:2). 0.5 ml saturated potassium iodide solution was added accurately, sealed with a stopper immediately, shaken for 1 minutes for extraction, added with 30 ml water and 5 ml starch indicator solution, and titrated immediately with sodium thiosulfate titration solution (0.01 mol/L) until the indigo blue in the upper aqueous phase disappeared. The titration results were corrected with the blank vehicle group.

The liquid concentrate was diluted with 5% dextrose injection so that the diluted solution contained 0.1 mg/mL nimodipine. The diluted solution was shaken to be even, and the stability of the diluted solutions was observed.

The results are as follows:

The concentrates: the nimodipine injections of Formulas 1-3 are light yellow, clear and transparent oily solutions. The color of the solutions gradually became darker with the increase of the usage amount of EPC. The color of the solutions after sterilization was significantly darker than that before sterilization, so that the solutions after sterilization failed to pass the color examination. The higher the EPC concentration, the more significant the increase of the color.

The diluted solutions: The nimodipine concentrates of Formulas 1-3 were diluted with 5% dextrose injection to the same concentration (0.1 mg/mL nimodipine). The diluted solutions were all transparent and colorless. There were no precipitates from the diluted solutions upon standing for 24 hours. There was no significant difference in the formulated dilutions of the three formulas.

The effect of EPC on the liquid concentrates of nimodipine and the dilutions thereof before and after sterilization: The effects of sterilization at 121° C. for 15 minutes on the nimodipine content and the related substances of the nimodipine concentrates, as well as on the pH value of the dilutions were investigated, and the results were shown in Table 3.

TABLE 3
The effects of sterilization on the nimodipine injections

| | | | Formula 1 | | Formula 2 | | Formula 3 | |
|---|---|---|---|---|---|---|---|---|
| Investigated items | | | Before | After | Before | After | Before | After |
| Item | | Standards | sterilization | sterilization | sterilization | sterilization | sterilization | sterilization |
| Description | | Light yellow clear | Light yellow | Light yellow | Light yellow | Dark yellow | Light yellow | Brown yellow |
| | | solution | clear solution | clear solution | clear solution | clear solution | clear solution | clear solution |
| pH | | 4.0-7.0 | 5.61 | 5.83 | 5.83 | 5.64 | 5.83 | 5.40 |
| Related | Impurity I | <0.5% | 0.009% | 0.017% | 0.039% | 0.108% | 0.068% | 0.085% |
| substances | Total | <1.0% | 0.186% | 0.203% | 0.271% | 0.647% | 0.280% | 0.887% |
| | impurities | | | | | | | |

| Peroxide value | NA | 0.09 | 0.19 | 0.11 | 0.15 | 0.19 | 0.10 |
|---|---|---|---|---|---|---|---|
| Free fatty acid | Volume of titration | 0.36 | 0.36 | 0.38 | 0.32 | 0.29 | 0.31 |
| | solution (0.01 mol/L | | | | | | |
| | sodium hydroxide) | | | | | | |
| | consumed, ml | | | | | | |
| API Content | 95.0%-105.0% | 100.80% | 102.53% | 100.87% | 102.50% | 101.94% | 100.92% |

The above experimental data showed that the amount of EPC has no significant effect on the pH value, nimodipine content and related substances of these formulations before and after sterilization. However, the color of nimodipine concentrates containing EPC became darker after sterilization, and the extent of color darkening after sterilization increases with the increase of the EPC concentration. In other words, the moist heat sterilization led to significant change in the description of nimodipine concentrates, indicating that the color change is caused by the phospholipids contained in the compositions.

### Example 2: Solubilizing Effects of Co-Solvents on Nimodipine

### Formulas 4-5: Solubilizing Effects of Co-Solvents on Nimodipine

TABLE 4
Solubilizing effects of co-solvents on nimodipine

| Components (g) | Formula 4 | Formula 5 |
|---|---|---|
| Nimodipine | 1.00 | 1.00 |
| HS15 | 45.00 | 45.00 |
| Propylene glycol | 54.00 | — |
| Glycerol | — | 54.00 |
| Total | 100 | 100 |

### Process:

1) HS15 was added into a conical flask, heated at 60° C. until HS15 completely melted, and shaken up and down to mix well for use;
2) The prescribed amount of nimodipine as the API was weighed into a beaker, added with the co-solvent propylene glycol or glycerol and the melted HS15, and stirred in a water bath at 60° C. to give a liquid concentrate;
3) The liquid concentrate was filled into vials (7 mL) (filling volume: 2 mL), and sterilized at 121° C. for 15 minutes;
4) The appearance of the concentrates was observed. The formation of solution upon dilution with 5% dextrose injection and the stability of the diluted solutions (the concentration of nimodipine is 0.1 mg/mL) were observed.

The experimental results are shown in the table below:

TABLE 5
Solubilizing effects of co-solvents on nimodipine

| Components | Formula 4 | Formula 5 |
|---|---|---|
| Appearance of the concentrate | Clear and transparent solution | layering |
| The formation of dilution | Clear | — |
| The stability of the | No drug precipitated | — |
| diluted solution | in 8 hours | |

As can be seen from the experimental results above, only the co-solvent propylene glycol made nimodipine form a clear and transparent concentrate. The concentrate after moist heat sterilization is a clear and transparent light yellow solution. When the concentrate was diluted with an aqueous vehicle, there was no precipitate in the formed diluted solution upon standing for 8 hours. However, when the co-solvent is glycerol, the formulated concentrate layered after sterilization, and the solubilizing effect of glycerol is unacceptable. This shows that not any co-solvent is suitable for nimodipine.

### Example 3: Solubilizing Effects of Non-Phospholipid Surfactants on Nimodipine

### Formulas 6-9: Solubilizing Effects of Non-Phospholipid Surfactants on Nimodipine

TABLE 6
Screening of solubilizing effects of non-
phospholipid surfactants on nimodipine

| Components (g) | Formula 6 | Formula 7 | Formula 8 | Formula 9 |
|---|---|---|---|---|
| Nimodipine | 1.00 | 1.00 | 1.00 | 1.00 |
| HS15 | 45.00 | — | — | — |
| Tween 80 | — | 45.00 | — | — |
| RH40 | — | — | 45.00 | — |
| ELP | — | — | — | 45.00 |
| Propylene glycol | 54.00 | 54.00 | 54.00 | 54.00 |
| Total | 100 | 100 | 100 | 100 |

### Processes:

1) HS15 and RH40 were added into separate conical flasks respectively, heated at 60° C. until HS15 and RH40 completely melted, and shaken up and down to mix well for use;
2) The prescribed amount of nimodipine as the drug substance was weighed into a beaker, added with the prescribed amounts of propylene glycol as well as HS15, Tween 80, RH40 or ELP, and stirred in a water bath at 60° C. to give a liquid concentrate;
3) The liquid concentrate was filled into vials (7 mL) (filling volume: 2 mL), and sterilized at 121° C. for 15 minutes;
4) The appearance of the concentrates was observed. The formation of solution upon dilution with 5% dextrose injection and the stability of the diluted solutions (the concentration of nimodipine is 0.1 mg/mL) were observed.

The experimental results are shown in the table below.

TABLE 7
Solubilizing effects of non-phospholipid surfactants on nimodipine

| Components | Formula 6 | Formula 7 | Formula 8 | Formula 9 |
|---|---|---|---|---|
| Appearance of the | Clear and | Clear and | Clear and | Clear and |
| concentrates | transparent | transparent | transparent | transparent |
| | solution | solution | solution | solution |
| The formation of | Clear | Clear | Clear | Clear |
| dilution | | | | |
| The stability of the | No drug | No drug | No drug | No drug |
| diluted solution | precipitated in 8 | precipitated in 8 | precipitated in 8 | precipitated in 8 |
| | hours | hours | hours | hours |

As can be seen from the experimental results above, the non-phospholipid surfactants HS15, Tween 80, RH40 and ELP made nimodipine form a clear and transparent concentrate. The concentrate after moist heat sterilization is a clear and transparent light yellow solution. When the concentrate was diluted with an aqueous vehicle, there was no precipitate in the formed diluted solutions upon standing for 8 hours.

### Example 4: Solubilizing Effects on Different Active Pharmaceutical Ingredients

### Formulas 10-14: Solubilizing Effects on Different Active Pharmaceutical Ingredients

TABLE 8
The study of solubilizing effects on different active ingredients

| Components (g) | Formula 10 | Formula 11 | Formula 12 | Formula 13 | Formula 14 |
|---|---|---|---|---|---|
| Nimodipine | 1.00 | — | — | — | — |
| Andrographolide | — | 1.00 | — | — | — |
| Posaconazole | — | — | 1.00 | — | — |
| Ginsenoside | — | — | — | 1.00 | — |
| Teniposide | — | — | — | — | 1.00 |
| HS15 | 45.00 | 45.00 | 45.00 | 45.00 | 45.00 |
| Propylene glycol | 54.00 | 54.00 | 54.00 | 54.00 | 54.00 |
| Total | 100.00 | 100.00 | 100.00 | 100.00 | 100.00 |

### Processes:

1) The prescribed amount of HS15 was added into a conical flask, heated at 60° C. until HS15 completely melted, and shaken up and down to mix well for use;
2) The prescribed amount of each of the active pharmaceutical ingredients was weighed into a beaker, added with the prescribed amounts of propylene glycol and HS15 orderly, and stirred in a water bath at 60° C.;
3) The liquid concentrate obtained in step 2) was filled into vials (7 mL) (filling volume: 2 mL), and sterilized at 121° C. for 15 minutes;
4) The appearance of the concentrates was observed.

The experimental results are shown in the table below.

TABLE 9
The study of solubilizing effects on different active pharmaceutical ingredients

| | Formula 10 | Formula 11 | Formula 12 | Formula 13 | Formula 14 |
|---|---|---|---|---|---|
| Appearance | Clear and | Turbid liquid, | Turbid liquid, | Turbid liquid, | Turbid liquid, |
| of the | transparent | no solution | no solution | no solution | no solution |
| concentrate | solution | was formed | was formed | was formed | was formed |

The results show that the system of the non-phospholipid surfactant in combination with the co-solvent used in the invention has different solubilizing effects on different active pharmaceutical ingredients. Unlike nimodipine, andrographolide, posaconazole, ginsenoside and teniposide failed to form clear and transparent solutions in said system. This suggests that the combination of HS15 and propylene glycol is not suitable for solubilizing all poorly soluble drugs.

### Example 5: Screening of Drug Load of Nimodipine Injections

### Formulas 15-31: Screening of Drug Load of Nimodipine Injections

TABLE 10
Screening of drug load of nimodipine injections

| Components | Formula | Formula | Formula | Formula | Formula | Formula |
|---|---|---|---|---|---|---|
| (g) | 15 (0%) | 16 (0.1%) | 17 (0.2%) | 18 (0.4%) | 19 (0.6%) | 20 (0.8%) |
| Nimodipine | 0.00 | 0.10 | 0.20 | 0.40 | 0.60 | 0.80 |
| HS15 | 45.45 | 45.41 | 45.36 | 45.27 | 45.18 | 45.09 |
| Propylene | 54.55 | 54.49 | 54.44 | 54.33 | 54.22 | 54.11 |
| glycol | | | | | | |
| Total | 100.00 | 100.00 | 100.00 | 100.00 | 100.00 | 100.00 |

| Components | Formula | Formula | Formula | Formula | Formula | Formula |
|---|---|---|---|---|---|---|
| (g) | 21 (0.9%) | 22 (1.0%) | 23 (1.1%) | 24 (1.2%) | 25 (1.5%) | 26 (2.0%) |
| Nimodipine | 0.90 | 1.00 | 1.10 | 1.20 | 1.50 | 2.00 |
| HS15 | 45.05 | 45.00 | 44.95 | 44.91 | 44.77 | 44.55 |
| Propylene | 54.05 | 54.00 | 53.95 | 53.89 | 53.73 | 53.45 |
| glycol | | | | | | |
| Total | 100.00 | 100.00 | 100.00 | 100.00 | 100.00 | 100.00 |

| | Formula 27 | Formula 28 | Formula 29 | Formula | Formula 31 |
|---|---|---|---|---|---|
| Components (g) | (3%) | (5%) | (10%) | 30(15%) | (20%) |
| Nimodipine | 3.00 | 5.00 | 10.00 | 15.00 | 20.00 |
| HS15 | 44.09 | 43.18 | 40.91 | 38.64 | 36.36 |
| Propylene glycol | 52.91 | 51.82 | 49.09 | 46.36 | 43.64 |
| Total | 100.00 | 100.00 | 100.00 | 100.00 | 100.00 |

### Processes:

1) The prescribed amount of HS15 was added into a conical flask, heated at 60° C. until HS15 completely melted, and shaken up and down to mix well for use;
2) The prescribed amount of nimodipine was weighed into a conical flask, added with the prescribed amounts of propylene glycol and HS15 orderly, sealed and stirred in a water bath at 60° C.;
3) The liquid concentrate obtained in step 2) was filled into vials (7 mL) (filling volume: 2 mL), and sterilized at 121° C. for 15 minutes;
4) The appearance and stability of the concentrates were observed.

The results showed that the concentrates formed by formulas 15-29 with 0%-10% of drug load were homogeneous and transparent solutions, in which the active pharmaceutical ingredient and the excipients were mixed uniformly without insoluble substances, and the color of the concentrates gradually changed from colorless and transparent to light yellow as the increase of the drug load; however, after fully stirring, there was still nimodipine insoluble in Formulas 30 and 31 with 15% and 20% of drug load, respectively.

The concentrates of Formulas 15-29 were left to stand at 25° C. for 1 hour. It was found that after the concentrates were left to stand for 1 h, crystals precipitated from the concentrate of Formula 29 with 10% of drug load, while no crystals precipitated in the concentrates of other Formulas.

Formulas 15-29 with 0%-10% of drug load were diluted with 5% dextrose injection so that the concentration of nimodipine in the diluted solutions was 0.1 mg/ml. The color of the formed diluted solutions gradually changed from colorless and transparent to light yellow as the increase of the drug load. The diluted solutions were left to stand at 25° C., and the experimental results showed that crystals precipitated from all diluted solutions prepared from the nimodipine concentrates with 3%, 5%, 10% of drug load in about 30 minutes, and crystals precipitated from all diluted solutions prepared from nimodipine concentrates with 1.2%, 1.5%, 2.0% of drug load in 24 hours, and the diluted solutions kept stable for a shorter time and the amount of the precipitate was increased as the increase of the drug load. No crystal precipitated from the diluted solutions prepared from the nimodipine concentrate with 0%-1.1% of drug load after they were left to stand for 96 hours, and there was no significant difference among individual groups, making them satisfy the requirements on intravenous injection. Accordingly, the concentration of nimodipine in the concentrates should be ≤1.1%.

Low drug load may result in low drug concentrations in the diluted solutions, bringing inconvenience to the clinical use. Thus, the preferred drug load is 0.5%-1.1%.

### Example 6: Screening of the Amount of HS15 in Nimodipine Injections

The inventors performed two screenings of the amount of HS15: the first screening was performed in the wide range (10%-60%), and the second screening was performed in the narrow range (40%-60%). The specific experiments are as follows:

TABLE 11
Screening of the amount of HS15 (in the wide range: 10%-60%)

| | Formula | Formula | Formula | Formula | Formula | Formula |
|---|---|---|---|---|---|---|
| Components (g) | 32 | 33 | 34 | 35 | 36 | 37 |
| HS15 content (%) | 10 | 20 | 30 | 40 | 50 | 60 |
| Nimodipine | 1 | 1 | 1 | 1 | 1 | 1 |
| HS15 | 10 | 20 | 30 | 40 | 50 | 60 |
| Propylene glycol | 89 | 79 | 69 | 59 | 49 | 39 |
| Total | 100 | 100 | 100 | 100 | 100 | 100 |

### Processes:

1) The prescribed amount of HS15 was added into a conical flask, heated at 60° C. until HS15 completely melted, and shaken up and down to mix well for use;
2) The prescribed amount of nimodipine was weighed into another conical flask, added with the prescribed amounts of propylene glycol and the heat-melted HS15, sealed, and stirred to dissolve in a water bath at 60° C. until a homogeneous oily solution was formed to give a liquid concentrate;
3) The liquid concentrate was purged with nitrogen gas, filled into vials (7 mL) (filling volume: 2 mL), and sterilized at 121° C. for 15 min.

The content of nimodipine and the related substances in the concentrates after sterilization were examined.

1 ml sterilized concentrate was diluted with 25 ml water for injection (pH 5.9-6.1) for the determination of the pH value.

The sterilized concentrate was diluted with 5% dextrose injection (the concentration of nimodipine in the diluted solution is 0.1 mg/mL), and shaken until it was uniform. The stability of the diluted solution was observed for 24 hours, including visual and microscopic observations.

The concentrates: The formulation with the HS15 content of less than 40% (by weight) was a light yellow turbid liquid, and nimodipine was not completely dissolved; the formulation with the HS15 content of 40%-60% was a clear and transparent light yellow solution, in which nimodipine was completely dissolved. Therefore, the amount of HS15 should be more than 40%.

The diluted solutions: The sterilized concentrates of Formulas 35 to 37 were diluted with 0.5% dextrose injection to the same concentration (the concentration of nimodipine in the diluted solutions was 0.1 mg/mL). All the diluted solutions were colorless and transparent, and there was no significant difference in the appearance of these diluted solutions. No crystals precipitated in the diluted solutions after standing at 25° C. for 24 hours, even when observed under a microscope.

TABLE 12
Screening of the amount of HS15 (in the narrow range: 40%-60%)

| | Formula | Formula | Formula | Formula | Formula | Formula | Formula | Formula | Formula | Formula | Formula |
|---|---|---|---|---|---|---|---|---|---|---|---|
| Components (g) | 38 | 39 | 40 | 41 | 42 | 43 | 44 | 45 | 46 | 47 | 48 |
| Contents of HS15 (%) | 40 | 42 | 44 | 46 | 48 | 50 | 52 | 54 | 56 | 58 | 60 |
| Nimodipine | 1 | 1 | 1 | 1 | 1 | 1 | 1 | 1 | 1 | 1 | 1 |
| HS15 | 40 | 42 | 44 | 46 | 48 | 50 | 52 | 54 | 56 | 58 | 60 |
| Propylene glycol | 59 | 57 | 55 | 53 | 51 | 49 | 47 | 45 | 43 | 41 | 39 |
| Total | 100 | 100 | 100 | 100 | 100 | 100 | 100 | 100 | 100 | 100 | 100 |

### Processes:

1) The prescribed amount of HS15 was added into a conical flask, heated at 60° C. until HS15 completely melted, and shaken up and down to mix well for use;
2) The prescribed amount of nimodipine was weighed into another conical flask, added with the prescribed amounts of propylene glycol and the heat-melted HS15 orderly, sealed, and stirred to dissolve in a water bath at 60° C. until a homogeneous oily solution was formed to give a liquid concentrate;
3) The liquid concentrates were purged with nitrogen gas, filled into vials (7 mL) (filling volume: 2 mL), and sterilized at 121° C. for 15 min.

The content of nimodipine and the related substances in the concentrates were examined.

1 ml sterilized concentrate was diluted with 25 ml water for injection (pH 5.9-6.1) for the determination of the pH value.

The sterilized concentrate was diluted with 5% dextrose injection (the concentration of nimodipine in the diluted solution is 0.1 mg/mL). The stability of the diluted solution was observed for 24 hours, including visual and microscopic observations.

### The Concentrates:

At a temperature higher than 25° C., the nimodipine concentrates of Formulas 38 to 48 were clear and transparent light yellow solutions, in which the active pharmaceutical ingredient and the excipients were mixed uniformly without insoluble substances. After standing and cooling to 25° C., the concentrates of Formulas 39 to 48 were unchanged and were still clear and transparent solutions, while the concentrate of Formula 38 became turbid and turned back to a clear and transparent light yellow solution under heating, and became turbid after cooling to 25° C. again. The inventors speculated that this might be caused by the poor solubilization due to the low amount of HS15.

In addition, in view of the lower solidification point of HS15 (22° C. to 30° C.), the higher the content of HS15, the higher the solidification temperature. A higher solidification temperature is unfavorable to quality control and clinical administration. Therefore, the inventors further investigated the effects of temperature on the physical state of the concentrates of Formulas 39 to 48. The experimental results showed that the solidification of the concentrates of Formulas 46-48 (56-60% HS15) onset at 18° C., the solidification of the concentrates of Formulas 42-45 (48-54% HS15) onset at 12° C., and the concentrates of Formulas 39-41 (42-46% HS15) kept unchanged at the temperature of as low as 10° C.

The diluted solutions: The sterilized concentrates of Formulas 38 to 48 were diluted with 0.5% dextrose injection to the same concentration (0.1 mg/mL). All the diluted solutions were colorless and transparent, and there was no significant difference in the appearance of these diluted solutions. No crystals precipitated in the diluted solutions after standing at 25° C. for 24 hours, even when observed under a microscope.

TABLE 13
The effects of the amount of HS15 on nimodipine concentrates before and after sterilization:

| | | Formula | Formula | Formula | Formula | Formula | Formula | Formula | Formula | Formula | Formula | Formula |
|---|---|---|---|---|---|---|---|---|---|---|---|---|
| Item | | 38 | 39 | 40 | 41 | 42 | 43 | 44 | 45 | 46 | 47 | 48 |
| HS15 content (%) | | 40 | 42 | 44 | 46 | 48 | 50 | 52 | 54 | 56 | 58 | 60 |
| Total | Before | 0.225 | 0.299 | 0.234 | 0.231 | 0.230 | 0.295 | 0.230 | 0.232 | 0.232 | 0.230 | 0.230 |
| impurities | sterilization | | | | | | | | | | | |
| (%) | After | 0.239 | 0.254 | 0.251 | 0.253 | 0.254 | 0.236 | 0.261 | 0.253 | 0.273 | 0.255 | 0.256 |
| | sterilization | | | | | | | | | | | |
| Nimodipine | Before | 97.86 | 100.46 | 101.22 | 102.11 | 100.25 | 101.03 | 100.48 | 101.06 | 100.54 | 102.23 | 102.49 |
| content (%) | sterilization | | | | | | | | | | | |
| | After | 98.02 | 102.08 | 101.08 | 100.86 | 101.90 | 101.34 | 101.26 | 102.85 | 101.91 | 103.32 | 102.56 |
| | sterilization | | | | | | | | | | | |
| pH | Before | 6.28 | 6.27 | 6.34 | 6.31 | 6.30 | 6.40 | 6.44 | 6.44 | 6.33 | 6.44 | 6.36 |
| | sterilization | | | | | | | | | | | |
| | After | 6.63 | 6.87 | 6.47 | 6.35 | 6.51 | 6.46 | 6.43 | 6.99 | 6.70 | 6.62 | 6.77 |
| | sterilization | | | | | | | | | | | |

For the liquid concentrates of nimodipine of Formulas 38 to 48, the heat moist sterilization did not affect the content of nimodipine, the related substances and the pH value.

The above experimental results showed that the amount of HS15 used in accordance with the invention is preferably 42%-60%.

HS15 causes a certain degree of hemolysis, which is lower than that caused by Polysorbate 80, and the serum histamine release level and the possibility of biological allergies caused by HS15 are also much lower than the latter (Lu H, Li J, Li M, et al. Systemic delivery of alpha-asarone with Kolliphor HS 15 improves its safety and therapeutic effect on asthma [J]. Drug Deliv, 2015, 22 (3): 266-275). YI Hong et al. (YI Hong, GAO Jin, YANG Hua et al., Overview of Quality Standards and Safety of Several Surfactants for Injection [J]. Chinese Journal of Experimental Traditional Medical Formulae, 2010, 16 (1): 115-119) reported that the LD50 of HS15 was 3.16 g/kg after intravenous injection of HS15 in mice. The results of guinea pig allergy test showed that compared with the polysorbate 80 group, the HS15 group had a lower level of plasma histamine release (at 60 min after intravenous injection, the serum histamine level was 8 nmol·L⁻¹ in the HS15 group and 247 nmol. L⁻¹ in the polysorbate 80 group), and a lower degree of hemolysis (the erythrocyte lysis was 1% in the HS15 group and 4% in the polysorbate 80 group after intravenous injection of the solubilizer at the concentration of 1%). Moreover, due to the low viscosity of HS15, HS15 has almost no effect on the viscosity of the drug solution, which greatly reduces the irritation of injection administration. However, an excessive dosage of HS15 can also cause hemolysis and allergic reactions.

Therefore, the amount of HS15 used in accordance with the invention is preferably 42%-50% (by weight), more preferably 44%-46% (by weight).

### Example 7: Screening of Terminal Sterilization in the Method for Preparing Nimodipine Concentrates

The inventors investigated the effect of sterilization conditions on the quality of nimodipine injections.

Formula (2022011901): nimodipine 1.00 g (1%), HS15 45.00 g (45%), propylene glycol 54.00 g (54%), total weight 100.00 g.

Process: 1) HS15 was heated at 60° C. in a flask until HS15 completely melted, and shaken up and down to mix well for use; 2) The prescribed amount of propylene glycol was weighed into a conical flask, added with the prescribed amounts of HS15 and nimodipine orderly, sealed, and magnetic stirred to dissolve in a water bath at 60° C. until a homogeneous solution was formed to give the liquid concentrate of nimodipine; 3) the liquid concentrate was filled into vials (7 mL) (filling volume: 2 mL), and divided into three groups. The first group was sterilized at 121° C. for 15 minutes, the second group was sterilized at 115° C. for 30 minutes, and the third group was sterilized by filtration.

The content of nimodipine and the related substances of the concentrates in each group were determined according to the methods described in Example 11.

The above three groups of samples and Nimotop® were diluted with 5% dextrose injection. The concentration of nimodipine is 0.1 mg/mL in the Diluted Solution 1, and 0.04 mg/mL in in the Diluted Solution 2. The pH values of the diluted solutions were determined, and the stability of the diluted solutions was observed.

TABLE 14
The effects of terminal sterilization on the nimodipine concentrates and diluted solutions

| | Concentrates | | | Diluted Solution 1 | | Diluted Solution 2 | |
|---|---|---|---|---|---|---|---|
| | Content of | Related substances (%) | | (0.1 mg/mL) | | (0.04 mg/mL) | |
| Sterilization | Nimodipine (%) | | Total impurities | pH | Stability of the | pH | Stability of the |
| method | 95.0%-105.0% | Impurity I (%) | (%) | 4.0-7.0 | solutions | 4.0-7.0 | solutions |
| Filtration | 100.11 | 0.006 | 0.231 | 6.24 | Drug precipitated | 5.00 | Drug |
| sterilization | | | | | at about 1 hour | | precipitated at |
| | | | | | | | about 1 hour |
| Moist heat | 101.49 | 0.014 | 0.242 | 6.29 | No drug | 5.02 | No drug |
| sterilization | | | | | precipitated in 8 | | precipitated in 8 |
| (115° C., 30 min) | | | | | hours | | hours |
| Moist heat | 101.26 | 0.014 | 0.245 | 6.37 | No drug | 5.01 | No drug |
| sterilization | | | | | precipitation in 8 | | precipitated in 8 |
| (121° C., 15 min) | | | | | hours | | hours |
| Originator's | 98.03 | 0.191 | 0.445 | 6.89 | Drug precipitated | 6.38 | Drug |
| product | | | | | at 0.5 hour | | precipitated at 0 |
| (Nimotop ®) | | | | | | | hour |

The applicant has surprisingly found that moist heat sterilization can significantly improve the stability of the diluted solutions. Without being bound by theory, the inventors speculated that the reason leading to this phenomenon may be that moist heat sterilization results in tighter molecular interaction of various substances contained in the concentrates.

“Technical Guidelines for Impurity Studies of Chemical Drugs” issued by Center for Drug Evaluation under National Medical Products Administration clearly indicates that the adverse effects occurred in the clinical use of a medicine are not only related to the pharmacological activity of the API, but also are related to the impurities of the medicine sometimes. Impurity I is the main impurity produced by the degradation of nimodipine, and is a structural analogue of compounds with hepatorenal toxicity, poses a safety risk, and thus is the major impurity for quality control. Therefore, less impurities will be better. It can be seen from the above table that no matter the liquid concentrates of nimodipine in accordance with the invention are subjected to moist heat sterilization at 115° C. for 30 min or at 121° C. for 15 min, the content of nimodipine and the related substances in the concentrate as well as the pH of the diluted solutions satisfy the limit requirements, and the amount of Impurity I and the amount of the total impurities in the liquid concentrates are all significantly less than that of Nimotop®.

### Example 8: Study on Whether the Method for Preparing the Liquid Concentrates of Nimodipine should be Protected from Light

Formula (2022011901-2): nimodipine 1.00 g (1%), HS15 45.00 g (45%), propylene glycol 54.00 g (54%), total weight 100.00 g.

Process: 1) HS15 was heated at 60° C. in a flask until HS15 completely melted, and shaken up and down to mix well for use; 2) The prescribed amount of propylene glycol was weighed into a conical flask, added with the prescribed amounts of HS15 and nimodipine orderly, sealed, and magnetic stirred to dissolve in a water bath at 60° C. until a homogeneous solution was formed to give the liquid concentrate of nimodipine; 3) the liquid concentrate was filled into vials (7 mL) (filling volume: 2 mL), and sterilized at 121° C. for 15 minutes. The process was performed under two conditions: natural light and protection from light.

The pH value, the content of nimodipine and the related substances of the concentrates were determined according to the methods described in Example 11.

TABLE 15
The effects of light on the concentrates of nimodipine

| | | | Liquid concentrates | | | |
|---|---|---|---|---|---|---|
| | With or | | | Related substances (%) | | |
| Batch number | without light | | Content (%) | Impurity I (%) | Total impurities (%) | pH |
| 2022011901 | natural light | Before sterilization | 100.11 | 0.006 | 0.231 | 6.24 |
| | | 115° C., 30 min | 101.49 | 0.014 | 0.242 | 6.29 |
| | | 121° C., 15 min | 101.26 | 0.014 | 0.245 | 6.37 |
| 2022011902 | protection from | Before sterilization | 99.77 | N/A | 0.225 | 6.12 |
| | light | 121° C., 15 min | 101.6 | N/A | 0.231 | 6.23 |

N/A: not be detected

The experimental data showed that the impurities in the concentrates prepared under the natural light and with protection from light satisfied the requirements, but the method with protection from light made the amount of Impurity I less. Therefore, the process with protection from light is preferred.

### Example 9: Study on Whether the Method for Preparing the Concentrate of Nimodipine Comprises Purging with Nitrogen Gas

The inventors investigated the effect of nitrogen gas on nimodipine injections. In Example 10, the effect of nitrogen gas on the related substances of nimodipine injections was studied in the liquid concentrate with the batch number 2022012201. The detailed experimental results are shown in the table below.

TABLE 16
The effects of nitrogen gas on the related substances
of the concentrates of nimodipine

| Batch | | | Total |
|---|---|---|---|
| number | Condition | Impurity I | impurities |
| 2022012201 | Purged with nitrogen gas | 0.004 | 0.137 |
| | Not purged with nitrogen gas | 0.015 | 0.160 |

The experimental data showed that the total impurities and Impurity I of the liquid concentrates in accordance with the invention prepared by the method with or without nitrogen purge all satisfied the requirements, but the content of the total impurities and the content of Impurity I in the concentrates prepared by the process with nitrogen purge are lower. Therefore, the method with nitrogen purge is more preferred.

### Example 10: Verification of the Small-Scale Process for Preparing the Composition in Accordance with the Invention

The inventors prepared two batches of nimodipine injections (batch numbers 2022012201 and 2022012202) with 600 vials for each batch, and conducted small-scale verification of the formula and process.

Formula: nimodipine 1.00 g (1%), HS15 45.00 g (45%), propylene glycol 54.00 g (54%), total weight 100.00 g.

Process: 1) HS15 was heated at 60° C. in a flask until HS15 completely melted, and shaken up and down to mix well for use; 2) The prescribed amount of propylene glycol was weighed into a conical flask, added with the prescribed amounts of HS15 and nimodipine orderly, sealed, and magnetic stirred to dissolve in a water bath at 60° C. until a homogeneous solution was formed to give the liquid concentrate of nimodipine; 3) the liquid concentrate was purged with nitrogen gas, and filled into brown and colorless vials; and 4) the liquid concentrates in the vials were subjected to moist heat sterilization at 121° C. for 15 minutes. The process was performed with protection from light.

Two batches of samples were prepared with the theoretical batch size of 600 vials, the number of qualified samples of 560 vials, and the yield of 93.3%.

### Example 11: Study on the Stability of the Composition in Accordance with the Invention

### Study on the Stability of the Concentrate:

According to the Guidelines for the Stability Testing of Drug Substances and Preparations in Section 9001 of the General Notices, Pharmacopoeia of the People's Republic of China 2020, Volume IV, the concentrates prepared in Example 10 were tested at a temperature of 60° C. or under the light with an intensity of 5000 Lux, and sampled at the end of the 5th, 10th and 30th days to examine the specified items.
1. Sample information: nimodipine injections (samples prepared in accordance with the invention) with the batch number 2022012201 (in brown and transparent vials); the originator's product with the batch number BXJC7A1 (Nimotop® in brown vials, and the sample in transparent vials were obtained by re-packing Nimotop® into the colorless vials).

### 2. Conditions for the Stability Tests:

TABLE 17
Conditions for the Stability Tests

| | | | Investigated |
|---|---|---|---|
| Item | Conditions | Time | items |
| Samples prepared in | At the high temperature of | Days 0, 5, 10, | Description, pH, |
| accordance with the | 60° C., or under the light of | 30 | related |
| invention (in brown vials) | 5000 Lux | | substances, |
| Samples prepared in | Under the light of 5000 Lux | | content of API |
| accordance with the | | | |
| invention (in transparent | | | |
| vials) | | | |
| Originator's product (in | At the high temperature of | | |
| brown vials) | 60° C., or under the light of | | |
| | 5000 Lux | | |
| Originator's product (in | At the high temperature of | | |
| transparent vials) | 60° C., or under the light of | | |
| | 5000 Lux | | |

### 3. Analysis Methods

### (1) Description

Method: visual inspection.

### (2) pH Value

1 ml of the concentrate was diluted with freshly boiled cold water in a ratio of 1:25 by volume for the pH value measurement. The pH value should be between 4.0 and 7.0.

### (3) Related Substances

Detection method: HPLC
Conditions for the HPLC:
Column: C18 column (column model: Ultinate XB-C18, XDB-C18, 125 cm, inner diameter 4.6 mm, particle size of packing 5.0 μm)
Column temperature: 40° C.
Detector: UV detector (detection wavelength 235 nm)
Mobile phase: methanol-tetrahydrofuran-water (20:20:60)
Flow rate: 2.0 mL/min
Injection volume: 20 μL
Run time: 40 min

System adaptability: in the chromatogram of the system adaptability solution, the resolution between the peak of nimodipine and the peak of Impurity I should be greater than 3.0.

### Detailed Procedures:

The tested concentrates were taken at each specified sampling time point. The samples were weighed accurately, and added with the mobile phase to dissolve and quantitatively dilute to prepare solutions containing about 1.6 mg nimodipine per mL, which were used as the test solutions. An appropriate amount of nimodipine reference substance was accurately weighed, and diluted with the mobile phase to give a solution containing about 3.2 μg nimodipine per mL as the Reference Solution A. Separately, an appropriate amount of nimodipine reference substance and Impurity I reference substance were taken, and added with the mobile phase to dissolve and dilute to prepare a mixed solution containing 1.6 μg nimodipine and 1.6 μg Impurity I, which was used as the system adaptability solution. The determination was performed by the high-performance liquid chromatography under the condition described above. The recorded time for chromatograms was 3 times the retention time of the main component peak. In the chromatogram of the system adaptability solution, the resolution between the peak of nimodipine and the peak of Impurity I should be greater than 3.0.

20 μL each of the test solutions and the reference solutions were measured accurately, and injected into the liquid chromatograph for recording chromatograms. In the chromatograms of the test solutions, if there is a chromatographic peak with the same retention time as Impurity I, the area of said peak shall be calculated according to the external standard method, and the amount of Impurity I shall not exceed 0.5% of the labeled amount of nimodipine. The peak area of other impurities shall be calculated using the area of the main peak of the Reference Solution A as control and according to the external standard method, and the amount of each of other impurities shall not exceed 0.2% of the labeled amount of nimodipine, and the total amount of impurities shall not exceed 1.0% of the labeled amount of nimodipine.

### (4) Content of Nimodipine

Detection method: HPLC
Conditions for the HPLC:
Column: C18 column (column model: Ultinate XB-C18, XDB-C18, 125 cm, inner diameter 4.6 mm, particle size of packing 5.0 μm)
Column temperature: room temperature
Detector: UV detector (detection wavelength 235 nm)
Mobile phase: methanol-tetrahydrofuran-water (20:20:60)
Flow rate: 2.0 mL/min
Injection volume: 20 μL
Run time: 40 min

System adaptability: The tailing factor of the peak of the reference solutions should be not greater than 2.0.

### Detailed Procedures:

The concentrates were weighed accurately, and added with the mobile phase to dissolve and dilute to prepare solutions containing about 0.2 mg nimodipine per mL, which were used as the test solutions. An appropriate amount of nimodipine reference substance was accurately weighed, and dissolved and diluted with the mobile phase to give a solution containing about 0.2 mg nimodipine per mL as the Reference Solution. 20 μL each of the test solutions and the reference solution were measured accurately, and injected into the liquid chromatograph for recording chromatograms. The peak areas were calculated according to the external standard method.

### 4. Experimental Results

TABLE 18
Conditions for the Stability Tests

| | | | | High temperature test at 60° C. | | | | | |
|---|---|---|---|---|---|---|---|---|---|
| | Investigated | Limits and | | (protection from light) * | | | Illumination test at 5000 Lux | | |
| Samples | items | requirements | Day 0 | Day 5 | Day 10 | Day 30 | Day 5 | Day 10 | Day 30 |
| Concentrates | Description | The sample should be a | Clear | Clear | Clear | Clear | Clear | Clear | Clear |
| in accordance | | clear viscous light | viscous | viscous | viscous | viscous | viscous | viscous | viscous |
| with the | | yellow liquid | light yellow | light | light | light | light | light | light |
| invention (in | | | liquid | yellow | yellow | yellow | yellow | yellow | yellow |
| brown vails) | | | | liquid | liquid | liquid | liquid | liquid | liquid |
| | pH value | The pH value shall in | 6.41 | 6.35 | 6.34 | 6.45 | 6.39 | 6.39 | 6.41 |
| | | the range of 4.0 to 7.0 | | | | | | | |
| | Related | Impurity I shall | N/A | 0.012 | 0.020 | 0.056 | 0.018 | 0.056 | 0.196 |
| | substances | be ≤0.5% | | | | | | | |
| | detected by | Each of other impurities | 0.048 | 0.048 | 0.047 | 0.048 | 0.048 | 0.048 | 0.049 |
| | HPLC | shall be ≤0.2% | | | | | | | |
| | | Total impurities shall | 0.113 | 0.143 | 0.103 | 0.223 | 0.088 | 0.128 | 0.337 |
| | | be ≤1.0% | | | | | | | |
| | Content of | The content of | 100.47 | 100.47 | 100.59 | 100.17 | 100.69 | 101.03 | 99.94 |
| | nimodipine | nimodipine | | | | | | | |
| | | (C₂₁H₂₆N₂O₇) in the | | | | | | | |
| | | samples shall be 95.0% | | | | | | | |
| | | to 105.0% of the | | | | | | | |
| | | labelled amount | | | | | | | |
| Nimotop ® | Description | The sample should be a | Clear | Clear | Clear | Clear | Clear | Clear | Clear |
| (in brown | | clear yellowish liquid | yellowish | yellowish | yellowish | yellowish | yellowish | yellowish | yellowish |
| vials) | | | liquid | liquid | liquid | liquid | liquid | liquid | liquid |
| | pH value | The pH value shall in | 6.91 | 6.93 | 6.97 | 7.04 | 6.91 | 6.91 | 6.89 |
| | | the range of 6.0 to 7.5 | | | | | | | |
| | Related | Impurity I shall | 0.064 | 0.089 | 0.071 | 0.234 | 0.265 | 0.268 | 0.826 |
| | substances | be ≤0.5% | | | | | | | |
| | detected by | Each of other impurities | 0.032 | 0.032 | 0.029 | 0.033 | 0.041 | 0.074 | 0.055 |
| | HPLC | shall be ≤0.2% | | | | | | | |
| | | Total impurities shall | 0.151 | 0.168 | 0.148 | 0.378 | 0.380 | 0.388 | 1.156 |
| | | be ≤1.0% | | | | | | | |
| | Content of | The content of | 98.48 | 98.45 | 98.72 | 98.43 | 98.20 | 98.13 | 96.85 |
| | nimodipine | nimodipine | | | | | | | |
| | | (C₂₁H₂₆N₂O₇) in the | | | | | | | |
| | | samples shall be 95.0% | | | | | | | |
| | | to 105.0% of the | | | | | | | |
| | | labelled amount | | | | | | | |
| Concentrates | Description | The sample should be a | Clear | — | — | — | Clear | Clear | Clear |
| in accordance | | clear viscous light | viscous | | | | viscous | viscous | viscous |
| with the | | yellow liquid | light yellow | | | | light | light | light |
| invention (in | | | liquid | | | | yellow | yellow | yellow |
| transparent | | | | | | | liquid | liquid | liquid |
| vials) | pH value | The pH value shall in | 6.41 | — | — | — | 6.44 | 6.36 | 6.40 |
| | | the range of 4.0 to 7.0 | | | | | | | |
| | Related | Impurity I shall | N/A | — | — | — | 2.973 | 4.005 | 12.316 |
| | substances | be ≤0.5% | | | | | | | |
| | detected by | Each of other impurities | 0.048 | — | — | — | 0.091 | 0.104 | 1.162 |
| | HPLC | shall be ≤0.2% | | | | | | | |
| | | Total impurities shall | 0.113 | — | — | — | 3.262 | 4.302 | 14.790 |
| | | be ≤1.0% | | | | | | | |
| | Content of | The content of | 100.47 | — | — | — | 97.63 | 90.49 | 76.25 |
| | nimodipine | nimodipine | | | | | | | |
| | | (C₂₁H₂₆N₂O₇) in the | | | | | | | |
| | | samples shall be 95.0% | | | | | | | |
| | | to 105.0% of the | | | | | | | |
| | | labelled amount | | | | | | | |
| Nimotop ® | Description | The sample should be a | Clear | Clear | Clear | Clear | Clear | Clear | Clear |
| (in | | clear yellowish liquid | yellowish | yellowish | yellowish | yellowish | yellowish | yellowish | yellowish |
| transparent | | | liquid | liquid | liquid | liquid | liquid | liquid | liquid |
| vials) | pH value | The pH value shall in | 6.85 | 6.86 | 6.84 | 6.89 | 6.84 | 6.82 | 6.85 |
| | | the range of 6.0 to 7.5 | | | | | | | |
| | Related | Impurity I shall | NA | NA | NA | NA | 7.758 | 22.123 | 40.954 |
| | substances | be ≤0.5% | | | | | | | |
| | detected by | Each of other impurities | 0.086 | 0.127 | 0.152 | 0.120 | 0.159 | 4.022 | 5.603 |
| | HPLC | shall be ≤0.2% | | | | | | | |
| | | Total impurities shall | 0.445 | 0.291 | 0.318 | 0.184 | 8.022 | 28.907 | 51.236 |
| | | be ≤1.0% | | | | | | | |
| | Content of | The content of | 98.03 | 99.02 | 98.77 | 95.83 | 84.60 | 57.42 | 3.06 |
| | nimodipine | nimodipine | | | | | | | |
| | | (C₂₁H₂₆N₂O₇) in the | | | | | | | |
| | | samples shall be 95.0% | | | | | | | |
| | | to 105.0% of the | | | | | | | |
| | | labelled amount | | | | | | | |

N/A: not detected;
—: not examined
* The high temperature tests were performed with protection from light. Therefore, there is no substantial difference between the package in brown vials and the package in transparent vials, and it is enough to only determine the stability data of one of the two groups.

The experimental results above showed:

Brown vials: After 30 days at 60° C. in the high temperature tests, the concentrates in accordance with the invention and the originator's product Nimotop® did not exhibit significant changes in various parameters, and all parameters thereof met the requirements. There was no significant difference between the concentrates in accordance with the invention and the originator's product Nimotop® except that after 30 days under the light of 5000 Lux in the illumination test, the amounts of the related substances in Nimotop® exceeded the acceptable limits, while all parameters of the concentrates in accordance with the invention satisfied the requirements.

Transparent vials: After 30 days under the light of 5000 Lux in the illumination tests, the concentrates in accordance with the invention and the originator's product Nimotop® did not exhibit significant changes in the description and the pH value, but the related substances thereof increased significantly and the content of nimodipine decreased significantly, which were due to the degradation of nimodipine caused by the light. The tests under this stress condition showed that after 30 days under the light of 5000 Lux, the content of nimodipine in the concentrates in accordance with the invention was 76.25%, while it decreased to 3.06% in Nimotop®, which meant almost complete degradation of nimodipine.

Therefore, the stability of the concentrates in accordance with the invention is significantly better than that of the originator's product Nimotop®.

### Study of the Stability of the Diluted Solutions:

The stability of the diluted solutions prepared from the concentrates in accordance with the invention and the originator's product (Nimotop®) were investigated.
1. Sample information: nimodipine injections with the batch number 2022012201
2. Conditions for the study on the stability of the diluted solutions:

TABLE 19
Conditions for the study on the stability of the diluted solutions

| | Dilution method (the concentration of nimodipine | | Investigated |
|---|---|---|---|
| Samples | in the diluted solutions is all 0.04 mg/mL) | Time | items |
| Originator's | 50 mL of the originator's product (50 mL:10 mg) was | 0, 2, 4, | Description, |
| product | added into 200 ml of 5% dextrose injection | 6, 8, | pH value, and |
| (Nimotop ®) | 50 mL of the originator's product (50 mL:10 mg) was | 24, 36, | status of |
| | added into 200 ml of 0.9% sodium chloride injection | and 48 | crystallization |
| Concentrates | 1 mL of the concentrate in accordance with the | hours | |
| in | invention (1 mL:10 mg) was added into 250 ml of 5% | | |
| accordance | dextrose injection | | |
| with the | 1 mL of the concentrate in accordance with the | | |
| invention | invention (1 mL:10 mg) was added into 250 ml of 0.9% | | |
| | sodium chloride injection | | |

3. Analysis methods

### (1) Description

Method: visual inspection

### (2) pH Value

The pH values of the diluted solutions were determined.

### (3) Status of Crystallization

Methods: visual inspection and electronic microscope.

### 4. Experimental Results

TABLE 20
Results of stability test of the diluted solutions

| Time (hour) | | | 0 | 2 | 4 | 6 | 8 | 24 | 36 | 48 |
|---|---|---|---|---|---|---|---|---|---|---|
| Originator's | Dextrose | Description | True | Turbid | Turbid | Turbid | Turbid | Turbid | Turbid | Turbid |
| product | injection | | solution | solution | solution | solution | solution | solution | solution | solution |
| Nimotop ® | | pH value | 6.38 | 6.36 | 6.36 | 6.36 | 6.36 | 6.18 | — | — |
| | | Status of | No crystals | Crystals | Crystals | Crystals | Crystals | Crystals | Crystals | Crystals |
| | | crystallization | | precipi- | precipi- | precipi- | precipi- | precipi- | precipi- | precipi- |
| | | | | tated | tated | tated | tated | tated | tated | tated |
| | Sodium | Description | True | Turbid | Turbid | Turbid | Turbid | Turbid | Turbid | Turbid |
| | chloride | | solution | solution | solution | solution | solution | solution | solution | solution |
| | injection | pH value | 5.82 | 5.81 | 5.81 | 5.81 | 5.81 | 5.66 | — | — |
| | | Status of | No crystals | Crystals | Crystals | Crystals | Crystals | Crystals | Crystals | Crystals |
| | | crystallization | | precipi- | precipi- | precipi- | precipi- | precipi- | precipi- | precipi- |
| | | | | tated | tated | tated | tated | tated | tated | tated |
| The | Dextrose | Description | True | True | True | True | True | True | True | True |
| concentrate | injection | | solution | solution | solution | solution | solution | solution | solution | solution |
| in | | pH value | 4.9 | 4.88 | 4.96 | 4.89 | 5.00 | 4.47 | 4.58 | 4.67 |
| accordance | | Status of | No | No | No | No | No | No | No | No |
| with the | | crystallization | crystals | crystals | crystals | crystals | crystals | crystals | crystals | crystals |
| invention | Sodium | Description | True | True | True | True | True | True | True | True |
| | chloride | | solution | solution | solution | solution | solution | solution | solution | solution |
| | injection | pH value | 5.92 | 5.89 | 5.81 | 5.80 | 5.88 | 5.85 | 5.85 | 5.03 |
| | | Status of | No | No | No | No | No | No | No | No |
| | | crystallization | crystals | crystals | crystals | crystals | crystals | crystals | crystals | crystals |

It can be seen from the experimental results in the table above that whether Nimotop® was diluted with 5% dextrose injection or 0.9% sodium chloride, nimodipine crystals crystallized rapidly upon dilution of Nimotop®. In clinical use, nimodipine must be infused slowly at a rate of 1-2 mg/h, otherwise the side effects could not be tolerated by the patients. Accordingly, the infusion time required for 10 mg of the drug is at least 5 hours. The originator's product comprises a high concentration of an organic solvent for solubilization, which will cause the API to crystallize upon dilution with dextrose injection and sodium chloride injection due to the decreased concentration of the organic solvent, resulting in increased adverse reactions and reduced efficacy. In order to solve this problem, a special three-way valve infusion set (FIG. 1) is necessary in the clinical use of Nimotop®, which brings inconvenience.

In contrast, after the liquid concentrate in accordance with the invention was diluted with 5% dextrose injection or 0.9% sodium chloride injection, the diluted solutions did not show significant changes in the description and the pH value after 48 hours at 25° C., and no crystals precipitated in the diluted solutions. Therefore, the liquid concentrate of nimodipine in accordance with the invention has overcome the drawbacks of the commercially available nimodipine injection, has good stability after dilution, and can be infused with an ordinary infusion set.

### DESCRIPTION OF DRAWINGS

FIG. 1 is a photograph of the three-way valve infusion set required for the intravenous infusion of Nimotop® in the clinical use. The port marked with the number “1” is the “venous end” and will be connected to the patient's vein. The port marked with the number “2” is the “fluid end with pump” and will be connected to the aqueous vehicle controlled by a pump. The port marked with the number “3” is the “nimodipine end” and will be connected to the nimodipine injection Nimotop®. The purpose of using this three-way valve is to infuse the nimodipine injection Nimotop® to the patient's vein immediately upon it is mixed with the aqueous vehicle, thereby avoiding the problem of crystallization of nimodipine caused by the standing of the diluted solution.

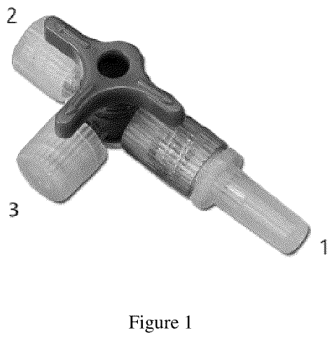

All patent and non-patent documents listed herein are incorporated by reference in their entirety as if each of them was all listed individually.

Although specific embodiments and examples are provided herein to illustrate the invention, they are not intended to limit the scope of the invention. Based on the present disclosure, other modified and equivalent solutions could be obviously obtained by those skilled in the art without departing from the spirit and essence of the invention, and those modified and equivalent solutions are within the scope of the invention.

1. A nimodipine composition free of phospholipid and ethanol, characterized in that the composition comprises:
(1) nimodipine;
(2) a non-phospholipid surfactant selected from polyoxyethylene castor oil, polyoxyethylene hydrogenated castor oil, polyoxyl 15 hydroxystearate, D-alpha-tocopherol polyethylene glycol 1000 succinate (TPGS), polysorbates, glyceryl monocaprylocaprate, or any mixture of two or more thereof;
(3) a co-solvent, which is propylene glycol,
wherein, based on the total weight of nimodipine, the non-phospholipid surfactant and the co-solvent, the amount of nimodipine is ≤1.1% w/w, preferably 0.5%-1.1% w/w, more preferably 0.8-1.1% w/w; the amount of the non-phospholipid surfactant is 42-50% w/w, preferably 44-46% w/w; and the remainder is the co-solvent, and
the composition is terminally sterilized by moist heat sterilization.

2. The composition according to claim 1, wherein the non-phospholipid surfactant is selected from polyoxyl 35 castor oil, pure polyoxyl 35 castor oil, polyoxyl 40 hydrogenated castor oil, polyoxyl 60 hydrogenated castor oil, polyoxyl 15 hydroxystearate, D-alpha-tocopherol polyethylene glycol 1000 succinate (TPGS), polysorbate 20, polysorbate 21, polysorbate 40, polysorbate 60, polysorbate 61, polysorbate 65, polysorbate 80, polysorbate 81, polysorbate 85, polysorbate 120, glyceryl monocaprylocaprate, or any mixture of two or more thereof.

3. The composition according to claim 2, wherein the non-phospholipid surfactant is polyoxyl 15 hydroxystearate.

4. The composition according to claim 1, wherein the composition further comprises a pH regulator and an antioxidant, and the pH regulator is preferably selected from one or more of citric acid, citrate (e.g., sodium citrate), maleic acid, tartaric acid, hydrochloric acid, sodium hydroxide, acetic acid, acetate (e.g., sodium acetate), phosphoric acid, and phosphate (e.g., sodium hydrogen phosphate, sodium dihydrogen phosphate, or sodium phosphate), the antioxidant is preferably selected from one or more of α-tocopherol succinate, ascorbyl palmitate, butylated hydroxyanisole, and butylated hydroxytoluene.

5. The composition according to claim 1, wherein the composition consists of nimodipine, the non-phospholipid surfactant and the co-solvent.

6. The composition according to claim 5, wherein the composition consists of nimodipine, polyoxyl 15 hydroxystearate and propylene glycol, and wherein based on the total weight of the composition, the amount of nimodipine is ≤1.1% w/w, preferably 0.5%-1.1% w/w, more preferably 0.8-1.1% w/w; the amount of polyoxyl 15 hydroxystearate is 42-50% w/w, preferably 44-46% w/w; and the remainder is propylene glycol, and
the composition is terminally sterilized by moist heat sterilization.

7. The composition according to claim 1, wherein the composition is prepared with protection from light.

8. The composition according to claim 1, wherein the composition is prepared by a method comprising a step of purging with nitrogen gas.

9. A method for preparing the composition according to claim 1, comprising the steps of: (1) mixing nimodipine, the non-phospholipid surfactant and the co-solvent to form a solution, if necessary, with heating to dissolve, (2) filling the resultant solution into a container, and (3) subjecting the filled solution to moist heat sterilization,
optionally, the method is performed with protection from light,
optionally, purging the solution obtained in step (1) with nitrogen prior to step (2),
preferably, the moist heat sterilization is performed at 115° C. for 30 min or at 121° C. for 15 min.

10. A solution obtained by diluting the composition according to claim 1 with an aqueous vehicle, wherein the aqueous vehicle is preferably a vehicle suitable for injection, for example, water for injection, 5% dextrose injection, 0.9% sodium chloride injection, lactated Ringer's solution, dextran 40 solution, hydroxyethyl starch 130/0.4 in sodium chloride injection, or hydroxyethyl starch 200/0.5 in sodium chloride injection.
